(19) United States
(12) Patent Application Publication
(10) Pub. No.: US 2021/0130778 A1
(43) Pub. Date: May 6, 2021

(54) METHOD FOR LARGE-SCALE PRODUCTION OF HUMAN ALLOSPECIFIC INDUCED-REGULATORY T CELLS WITH FUNCTIONAL STABILITY IN THE PRESENCE OF PRO-INFLAMMATORY CYTOKINES WITH THERAPEUTIC POTENTIAL IN TRANSPLANTATION

(71) Applicant: UNIVERSIDAD NACIONAL AUTONOMA DE MEXICO, CIUDAD DE MEXICO (MX)

(72) Inventors: Maria Gloria SOLDEVILA MELGAREJO, Mexico City (MX); Evelyn Katy ALVAREZ SALAZAR, Mexico City (MX); Josefina ALBERU GOMEZ, Mexico City (MX); Arimelek CORTES HERNANDEZ, Mexico City (MX)

(21) Appl. No.: 17/085,284

(22) Filed: Oct. 30, 2020

(30) Foreign Application Priority Data
Oct. 30, 2019 (MX) MX/A/2019/012911

(51) Int. Cl. C12N 5/0783; A61K 35/17

(52) U.S. Cl. CPC C12N 5/0637; A61K 35/17; C12N 2502/1121; C12N 2501/2306; C12N 2501/515; C12N 2501/25

(57) ABSTRACT

A methodology to obtain in vitro large numbers of human induced regulatory T cells with specificity to the donor antigen, with a phenotype and stable suppressor function in the presence of pro-inflammatory cytokines, through of co-cultures of monocyte-derived dendritic cells and T cells “ naïve ”, both from genetically unrelated individuals (donor and recipient) is disclosed. The cells obtained with the present method are of CD4, CD25, CTLA-4 and FOXP3+ phenotype and show a specific suppressor function on donor antigen-specific T lymphocytes. These cells maintain their phenotype and stable suppressive function in presence of pro-inflammatory cytokines TNF-α and IL-6. The stability and the number obtained make them candidates as therapeutic tools for transplantation.

### CROSS-REFERENCE TO RELATED APPLICATION

This Application claims priority from Mexican Patent Application No. MX/a/2019/012911, filed Oct. 30, 2019, the contents of which is incorporated herein by reference.

### TECHNICAL FIELD

The present invention provides a new method for de novo generation and expansion of human allospecific regulatory T cells for the induction of transplant tolerance and its use as an alternative or complementary therapy to conventional immunosuppressive drugs.

### BACKGROUND OF THE INVENTION

Organ transplantation is the best therapeutic alternative in patients with terminal and irreversible organ dysfunction. The use of immunosuppressive drugs have successfully reduced the incidence of acute rejection episodes, but their lack of selectivity can lead to side effects that are responsible for chronic rejection [1]. For this reason, the goal of transplantation is the induction of antigen-specific tolerance to reduce the chronic use of immunosuppressive drugs [2].

However, an important problem is the high frequency of alloreactive T cells in the general repertoire and the absence of their thymic deletion. Therefore, the participation of extra-thymic mechanisms seems to be the most important factor in the long-term acceptance of the allograft (a graft transplanted between two genetically different members of the same species) and among them, immune regulation by regulatory T cells is postulated as the predominant mechanism. Regulatory T cells play an important role in the maintenance of tolerance to self-antigens and the induction of transplantation tolerance [3,4], which could influence the fate and long-term acceptance of the transplanted organ [5].

Regulatory T cells (Tregs) are a subtype of CD4⁺ T lymphocytes that play a crucial role in the control of autoimmune diseases and the homeostasis maintenance, preventing the development of immunopathologies [6]. These cells are characterized by the constitutive expression of CD25 (a chain of IL-2 receptor) and the transcription factor FOXP3, defined by various authors as the master regulator for the development and function of Tregs, and considered as the main molecular marker of this subpopulation [7, 8].

These cells can be generated in the thymus, known as thymic regulatory T cells (tTregs, previously referred to as natural Tregs [nTregs]), and require the combination of strong antigenic and high costimulation signals for their development. But also, they can be generated in the periphery from “naïve” T lymphocytes after encounter self or foreign antigens, under limiting costimulation conditions and under an immunosupressor microenvironment during the time of activation; these cells are called peripheral Tregs (pTregs) if they are generated in vivo or induced Tregs (iTregs) if they are generated in vitro [9]. Both subpopulations of thymic and peripheral Tregs have a demethylated TSDR region (located in CNS2 of the FOXP3 gene) [10, 11], which is responsible for the maintenance of FOXP3 expression and therefore, they share a global pattern of gene expression, stability and phenotype. In contrast, iTregs show partial methylation of this region, which is associated with their lower stability under inflammation conditions.

Although the ability of regulatory T cells to suppress allograft rejection has been demonstrated in experimental models, the percentage of these cells (1-3% of human CD4⁺ T cells) is lower than of effector alloreactive T cells. This has motivated the development of a wide number of strategies that allow their ex vivo expansion, with the intention of reinfusing them into patients.

Initially, high expression of CD25 was used as the main marker to isolate regulatory T cells, however in humans this may include a contaminating fraction of activated conventional T cells. Therefore, other markers have been proposed to isolate more pure populations of Tregs with a greater suppressive function and epigenetic stability; among them, CD45RA [12], the ectoenzyme CD39 [13] and the glycoprotein containing leucine rich repeats (GARP), which is highly expressed by activated Tregs [14]. Additionally, the low expression of CD127 [15] or the non-expression of the integrin a chain CD49d and serine protease CD26 [16, 17] are other characteristic features that allow to distinguish Tregs from effector T cells. High purity of Tregs is essential for their efficient expansion, however the need of extensive stimulation to achieve the adequate number for their clinical use involves the risk of impairing Treg function [18]. Many clinical trials have used thymic Tregs as cellular therapy for the treatment and prevention of graft versus host disease, with no apparent functional toxicity. However, the main obstacle in the attempt to obtain sufficient cell numbers of Tregs, is the lack of specific Treg markers for their adequate purification. On the other hand, Tregs isolated from patients may carry intrinsic defects and therefore, could interfere with their suppressive function, making them not suitable for immunotherapy [19].

On the other hand, “naive” T cells (CD45RA+) are very abundant in the organism and thus, several groups have tried to generate iTregs in vitro, by converting CD4⁺CD25⁻ CD45RA⁺ T cells into FOXP3+ T cells. One of the main cytokines used to induce FOXP3 expression in CD4⁺CD25⁻CD45RA⁺ is TGF-β, with or without retinoic acid and rapamycin (RAPA) [20], in the presence of IL-2.

tTregs cells are selected in the thymus after self-antigen recognition with relatively high avidity, whereas pTregs require suboptimal signals and low costimulation during peripheral antigen recognition. These characteristics led to postulate that most tTregs would directed towards self-antigens, being specially relevant in the prevention of autoimmunity, while pTregs would regulate the response towards certain foreign antigens, such as those found in the intestinal microbiota and the fetus during pregnancy [21, 22].

However, the functions of thymic and peripheral Tregs are not redundant and both subpopulations are required to effectively suppress the immune response [23, 24]. Despite this, there are differences between them concerning their regulatory mechanisms. For example, some reports highlight the superiority of in vitro generated Tregs to migrate to inflammation sites compared to tTregs, indicating that those are primarily active in inflammatory tissues and regulate inflammation by direct suppression of endothelial activation and leukocyte recruitment [25]. Besides, studies in murine models indicate that TGF-β-induced iTregs could be more effective than tTregs under an inflammatory environment since they were resistant to conversion to Th17 in the presence of IL-6 [26], 2008 #1065}, while tTregs did not suppress TH17 cell-mediated inflammation in autoimmune gastritis [27] or collagen-induced arthritis [28].

Importantly, a report highlights the critical role of pTregs in the induction of allograft tolerance, since it was reported in a corneal transplant model, that pTregs (identified as Nrp-1⁻) were more efficient than tTregs in preventing allograft rejection [29]. These results suggest that pTregs could have a great advantage in the treatment of autoimmune and inflammatory diseases, as well as in the regulation of alloimmune responses in a transplant setting.

To date, there are few published protocols on in vitro generation of human Tregs, most of which use polyclonal stimulators and short-term cultures (1 to two weeks). Some of these studies do not analyze the methylation pattern of the FOXP3 gene [30, 32] while others report FOXP3 methylation or the lose of FOXP3 expression in response to antigenic re-stimulation [33, 35]. There are only a few protocols in which allospecific Tregs are generated. For example, Wenwei Tu et al, used B-CD40L cells (stimulated via CD40 by co-culturing with NIH3T3 cells transfected with CD40 ligand) for the generation of iTregs. Although they obtained up to 8.3×10⁶ of Tregs per million of “naïve” and 92% purity after 21 days of culture, the iTregs cells required to be expanded by the addition of B-CD40L cells per week [36], affecting the purity of the cellular product, due to contamination with B cells and the remaining transfected NIH3T3 cells. Moreover, none of the reported studies evaluated the stability of generated iTregs under inflammatory conditions, as it would be expected to happen in a transplant setting.

Dendritic cells (DCs) are antigen presenting cells with a great capacity to activate naïve T cells, making them ideal candidates for the generation of antigen-specific Tregs [37]. Depending on the type of signals received, immature DCs can differentiate into immunogenic or tolerogenic DCs [38]. It has been reported that in vitro expansion of tTregs is favored by the use of mature DCs [39], while the conversion of naive T cells to iTegs is favored by immature DCs [40] or under certain specific conditions of maturation [41].

A study compared the efficiency in Tregs generation by B-cells activated via CD40 (CD40-B) and immature DC derived from monocytes and concluded that CD40-B were better to induce and expand Tregs, and these were better suppressors than those induced by immature DC [42] although the differences observed between both allogeneic APCs were attributed to their ability to secrete IL-2.

In the present invention, the Treg-induction method has been optimized, by adding TGF-β, IL-2 and ATRA (by its name alpha-trans retinoic acid) to the co-cultures between DCs and “naïve” T cells, in order to improve the efficiency of iTreg generation by allogeneic DCs.TGF-β and IL-2 are essential for pTregs differentiation, where IL-2 signaling promotes FOXP3 expression through direct binding of STAT5 to the promoter and FOXP3 CNS2 region [43], and ATRA increases SMAD3 signaling promoted by TGF-β and inhibits the production of pro-inflammatory cytokines by memory T cells [44, 45].

On the other hand, with the methodology developed in the present invention, a large number of allospecific regulatory T cells were generated and expanded, using a three-step strategy: first, de novo generation of allospecific iTregs by co-cultures between naïve T cells and monocyte-derived DC; second, the iTreg isolation by “FACS sorting” and third, the expansion of purified iTregs for six weeks by in vitro polyclonal stimulation. This same methodology was reported in another work in which thymic Tregs were expanded for clinical use [46] obtaining an expansion of 100 to 1600 times. Also, other strategies include the continuous addition of allogeneic APC to the co-cultures for tTreg expansion or iTreg generation [47] [36].

With the invention described below, it is possible to in vitro generate induced regulatory T cells (iTregs) through stimulation of human “naïve” T cells with donor antigens, which can be expanded on a large scale, maintaining their phenotype and antigen-specific suppressive function under inflammatory conditions.

### BRIEF DESCRIPTION OF THE FIGURES

FIG. 1. This figure shows that Mo-DCs express markers representative of the DCs lineage such as CD86, CD11c, and HLA DR, but low levels of CD14, a characteristic monocyte marker (A). The Mo-DCs generated are capable of inducing an allogenic response when co-cultured with CD3⁺ T cells from an individual with a different genetic background (B).

FIG. 2. Mo-DCs were co-cultured with “naive” T cells in three different conditions of iTregs generation. Among them, no differences were observed in the frequency of CD4⁺CD25⁺FOXP3⁺ population (A). However, co-cultures with TGF-β1 and ATRA induce high levels of FOXP3 (B, bar graph, left), besides generating a greater number of iTregs (B, bar graph, right).

FIG. 3. Schematic representation of protocol for iTreg generation and expansion (A). Large scale production of iTregs cells (B, dot plot, left) and of viability percentage (B, dot plot, right) after 6 weeks of expansion.

FIG. 4. The majority of specific iTregs cells expanded for 6 weeks are CD4+ CD25⁺FOXP3⁺.

FIG. 5. Allo-iTregs cells polyclonally for 6 weeks maintain CD25 expression (A, dot plot, top), express high levels of FOXP3 reaching their maximum level at the fourth week (A, dot plot, bottom), and are primarily CTLA-4⁺ (B).

FIG. 6. The addition of the pro-inflammatory cytokines IL-6 and TNFα does not affect the frequency of CD25⁺FOXP3⁺ (A) and CTLA-4⁺ (B) cells in expansion cultures.

FIG. 7. Maintenance of CD25 (A) and FOXP3 (B) levels in the expansion cultures of iTregs in the presence of the pro-inflammatory cytokines IL-6 and TNFα.

FIG. 8. Allospecific iTregs cells expanded for 6 weeks suppress the proliferation of alloantigen-specific CD3⁺ T cells, which is not affected by the presence of pro-inflammatory cytokines.

FIG. 9. The suppressive function of expanded allospecific iTregs cells is associated with IL-10 and IFN-γ production and a decrease in IL-2 levels.

FIG. 10. Expanded allospecific iTregs cells require RAPA to maintain their CD25⁺FOXP3⁺ (A) phenotype and FOXP3 (B) expression.

### DETAILED DESCRIPTION OF THE INVENTION

The present invention describes an in vitro method to generate and expand large numbers of allospecific regulatory T cells with a stable phenotype and suppressive function under inflammatory conditions. Their stability, specificity, and numbers achieved, for the first time, makes them candidates for cellular immunotherapy and lays the foundations for the development of new strategies for in vitro large-scale generation of iTregs.

The present invention provides a method for obtaining human induced allospecific CD25⁺CTLA-4⁺FOXP3⁺ regulatory T cell populations with a stable phenotype and function in the presence of pro-inflammatory cytokines (TNF-α and IL-6). The allospecificity of human induced regulatory T cells obtained with the method of the present invention are evaluated for their ability to suppress the proliferation and cytokine production of donor CD3⁺ allogeneic T lymphocytes.

The method of the present invention to in vitro generate and expand regulatory T cells considers a three-step strategy: first, obtaining allospecific Tregs cells from the co-culture between “naïve” T cells from an individual (donor 1) with immature DCs from another individual (donor 2); second, the isolation of the iTregs obtained at the first step; and third, the polyclonal expansion of the cells obtained at the second step.

Immature DCs were derived from monocytes (Mo-DCs) cultured for 8 to 10 days in the presence of GM-CSF (50 ng/ml) and IL-4 (50 ng/ml) and were subsequently identified by low MHC class II expression and the presence of costimulatory molecules. For the present invention, donor 1 and 2 terms are healthy individuals, genetically unrelated, so the degree of compatibility between the two individuals is low or null. After 8 to 10 days of culture, non-adherent, large and long cells, with numerous projections from their membrane were identified, which expressed characteristic DC surface markers, and induced a significant proliferative response of allogeneic CD3⁺ T cells.

To generate allospecific iTregs, “naïve” T cells from an individual (donor 1) were co-cultured with immature DCs from another individual (donor 2), which in a transplant scenario would represent the recipient and donor, respectively. The co-culture was carried out in the presence of 5 to 10 ng/mL of TGF-β1, 10 nM of ATRA and 50 to 100 U/mL of IL-2. The co-cultures between “naïve” T cells and Mo-DCs were carried out in a ratio of 10:1, which favored FOXP3 induction and the proliferation of the induced allospecific Tregs.

Allospecific Tregs cells were identified based on the CD25⁺ and CFSE⁻ markers, corresponding to those activated and proliferating T cells that recognize the antigen. These cells cannot be isolated based on FOXP3 expression, as this marker is a transcription marker only detected after intracellular staining. Thus, CD25^{very high} positive cells were purified, which positively correlates with FOXP3 upregulation [48], to distinguish Tregs from the rest of activated T cells, which express lower levels of surface CD25.

The isolated cells were expanded for 6 weeks with anti-CD3/CD28 beads at a ratio of 1:1 to 1:2 (bead: cell), TGF-β1 at a concentration of 5 to 10 ng/mL, IL-2 at a concentration of 50 to 100 U/mL and 100 ng/mL of RAPA for 4 days (expansion phase). After 4 days, the beads were separated and the cells were left alone in the culture medium in the presence of 50 U/mL of IL-2 for 3 days (resting phase). This same scheme (expansion/resting) was repeated for 6 weeks. Throughout the expansion, an increase in viability was obtained, reaching 90% in the 6th week. It is important to note that after each expansion cycle the cells were maintained for 3 days in resting conditions, with only IL-2, to avoid their activation-induced cell death as a result of overstimulation, as well as to reduce activation-dependent CD25/FOXP3 upregulation, which could lead to overestimate the frequency of iTregs generated in our assays.

With the methodology developed in the present invention, an expansion of 230 thousand times the initial number was achieved, which is the highest achieved in the generation of induced allospecific Tregs reported so far. Specifically, from the initial 2×10⁴ allospecific iTregs cells, 4.6×10⁸ allospecific iTregs were obtained at the end of the 6th week of expansion.

According to clinical trials using thymic Tregs expanded with anti-CD3/CD28 beads, the average number of cells required to obtain an adequate suppression is 10-20×10⁶ cells/kg per patient [49]. Therefore, for a 70 kg patient an approximate of 700-1,400×10⁶ Tregs are needed; with the number reached in the present invention, it would be possible to infuse the patient with several doses of these cells. Importantly, it was estimated that the use of specific, rather than polyclonal Treg cells would allow to reduce the dose of Tregs required to achieve the same level of immunosuppression [47].

### EXAMPLES

### Example 1

### Generation and Characterization of Monocyte-derived Dendritic Cells (Mo-DCs)

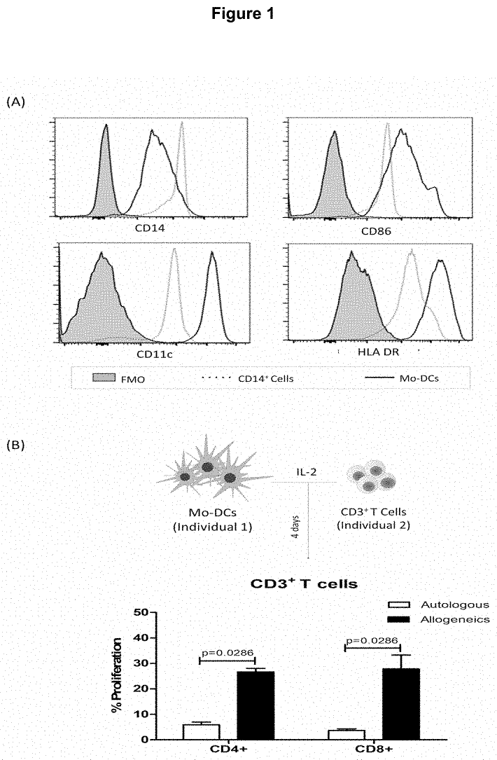

DCs were derived from CD14⁺ monocytes isolated from buffy coat preparations of peripheral blood from healthy donors (donor 1), which were provided by the Blood Bank of Instituto Nacional de Enfermedades Respiratorias. For this aim, peripheral blood mononuclear cells (PBMCs) were isolated by density gradient centrifugation over Ficoll-Paque™ (GE Healthcare), according to the manufacturer's instructions. A proportion of PBMCs were resuspended in cold freezing medium (10% DMSO and 90% Fetal Bovine Serum) at a concentration of 10⁷ cells/mL, stored for 24 hours at −70° C. and then transferred to liquid nitrogen for long-term storage; for additional functional assays, PBMCs were thawed in a 37° C. water bath and were collected in RPMI medium supplemented with 10% FBS, washed twice and resuspended in culture medium. CD14⁺ monocytes were purified from freshly isolated PBMCs using the Human CD14 MicroBeads kit (Miltenyi Biotec), according to the manufacturer's instructions. Isolated CD14⁺ monocytes were cultured in RPMI medium supplemented with 10% human serum and stimulated with IL-4 (50 ng/mL) and GM-CSF (50 ng/mL) for 8 days; on days 3 and 5, the culture medium and cytokines were refreshed. At the end of the culture Mo-DCs were harvested, washed twice with culture medium and irradiated with 3000 rads before the functional assays. To characterize the Mo-DCs, these cells were stained with anti-CD14, anti-CD11c, anti-HLA-DR and anti-CD86 monoclonal antibodies for 20 min at 420 C. in the dark, washed twice with FACS buffer and acquired on the Attune Flow Cytometer (Themor Fisher); the data were analyzed with FlowJo vX.0.7 software (FIG. 1A). To evaluate the ability to stimulate the alloresponse in vitro, Mo-DCs were co-cultured with allogeneic CD3⁺ T cells (labeled with CFSE) for 5 days, and then cells were stained with monoclonal antibodies, acquired on the flow cytometer and the percentage of proliferation was determined by CFSE dilution on gated CD4⁺ or CD8⁺ T cells (FIG. 1).

### Example 2

### Allogeneic Co-Culture between Naive T Cells and Monocyte-Derived Dendritic Cells (Mo-DCs)

PBMCs were obtained from healthy donors and purified through a Ficoll-Paque™ Plus (GE Healthcare) gradient. 50×106 de PBMCs were incubated with anti-CD4, anti-CD25 y anti-CD45RA for 20 min at 4° C. Then, cells were washed and resuspended in PBS 1× and CD4+CD25−D45RA+ (“naïve” T cells) were purified on a FACs Aria I cell sorter (BD Biosciences), collected RPMI/20% FBS media and stained with the fluorescent dye CFSE in PBS×1. Then, “naïve” T cells were resuspended in OpTmizer™ CTS™ T-Cell Expansion medium(Gibco), co-cultured with Mo-DCs from example 1 in a ratio 1:10 (1: “naïve” T cell). Three conditions were evaluated for iTreg generation: (1) 50-100 ng/mL de TGF-β1 alone; (2), 50-100 ng/mL TGF-β1+10 nM ATRA and (3) 50-100 ng/mL TGF-β1+10 nM ATRA and 100 ng/mL de RAPA, all in the presence of 50-100 U/mL IL-2, in 96 well plates for 7 days. Both cell sources were from different donors (allogenic co-culture).

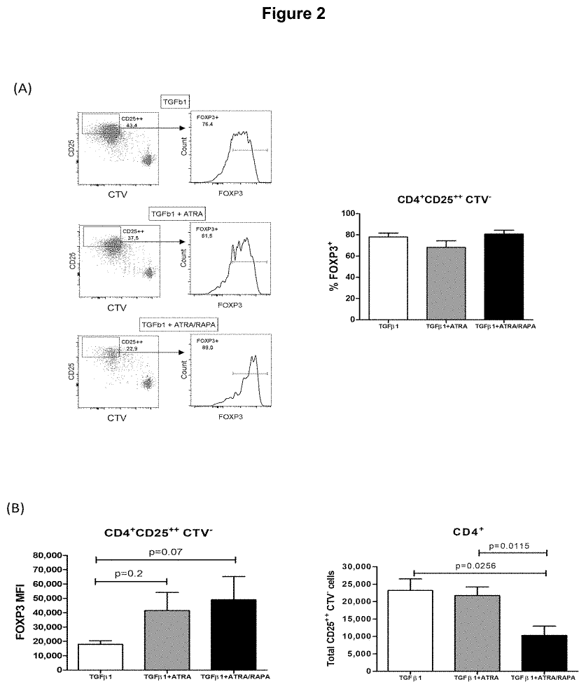

The three culture conditions were able to generate iTregs, as evidenced by the expression of the CD25 and FOXP3 markers (FIG. 2A, histogram and bar graph), although FOXP3 expression was higher in conditions 2 and 3 compared to condition 1 (FIG. 2B, bar graph, left). However, a better cell expansion was achieved in condition 2 compared to 3 (FIG. 2B, bar graph, right), therefore this condition (2) was chosen for the generation of allospecific iTregs.

### Example 3

### Isolation of iTregs Based on the CD4⁺CD25^{hi} Markers

After 7 days of culture, the proliferating CD4⁺CD25^{hi} cells were isolated from the co-cultures between “naive” T cells and Mo-DCs. For this, the cells were stained with anti-CD4 and anti-CD25 antibodies and sorted in the FACS Aria I cell sorter (BD Biosciences) to isolate the proliferating CD4⁺CD25^{hi}CFSE⁻ cells (allospecific induced regulatory T cells) and the non-proliferating CD4⁺CFSE⁺, which were co-cultured for 7 days in the presence of irradiated Mo-DCs under the conditions specified in Example 1. The isolated cells were collected in RPMI medium supplemented with 20% FBS, washed and resuspended in OpTmizer™ CTS™ T-Cell Expansion culture medium (Gibco) supplemented with only 50 U/mL IL-2 for 3 days (resting) before polyclonal expansion.

### Example 4

### Expansion of Allospecific Induced Regulatory T Cells

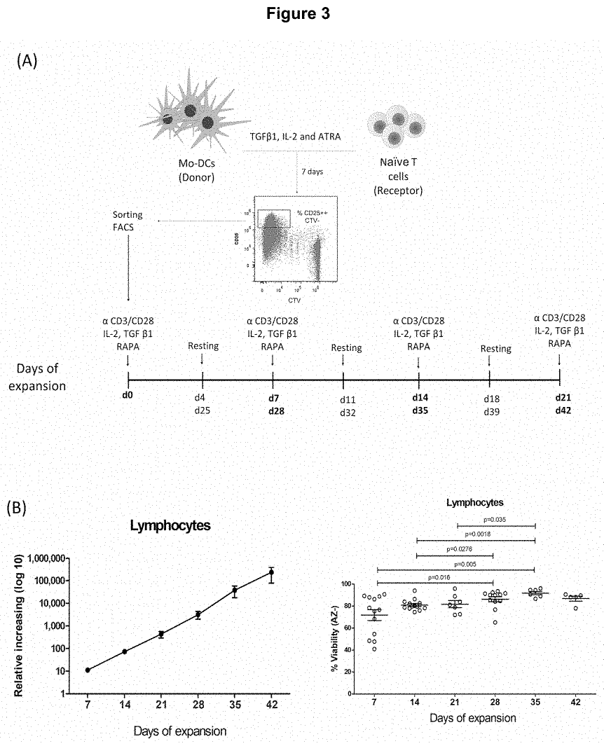

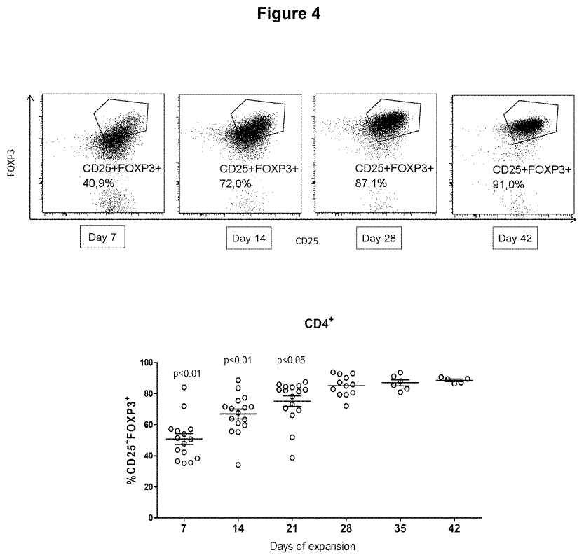

On the third day, the cells from example 3 were washed and cultured in the presence of the following stimuli: anti-CD3/CD28 beads, in a ratio of 1:1 to 1:2 (beads: cell), 5-10 ng/mL of TGF-β1, 50-100 U/mL of IL-2 and 100 ng/mL RAPA for 4 days (expansion), with a re-stimulus of IL-2 (50-100 U/ml) on day 2. After 4 days of expansion, the beads were removed with DynaMag (Gibco), cells were washed twice with culture medium and rested for three days in expansion medium containing 50 U/mL of IL-2 (resting). This scheme was repeated for six weeks (FIG. 3A) and at the end of the expansion, the cells reached a relative increase of 230 thousand times the initial number (FIG. 3B, left) with a viability of 90% (FIG. 3B, right). Furthermore, the expanded cells presented a CD25+FOXP3+ phenotype, whose frequency increased from the first week of expansion until reaching a maximum close to 90% at the fourth week of culture (FIG. 4).

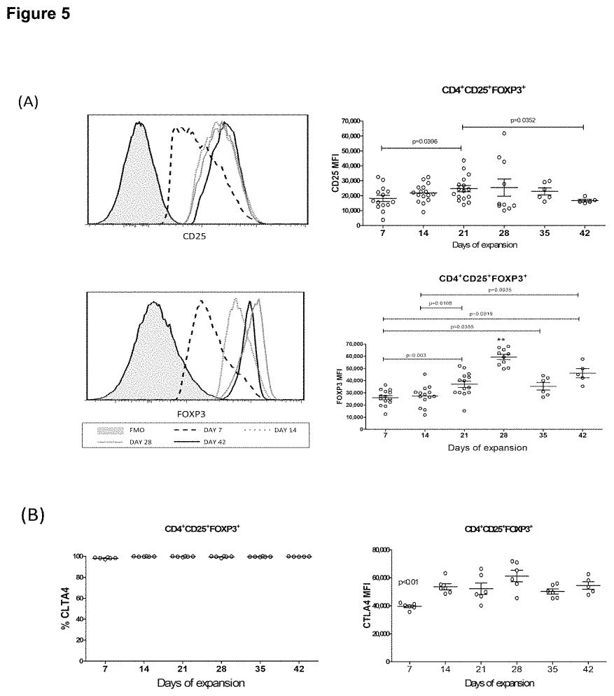

CD25 and CTLA-4 expression was maintained throughout the iTreg expansion and was not affected by the continuous re-stimulation. Furthermore, FOXP3 increased until reaching a maximum expression level at the fourth week of expansion (FIG. 5A and FIG. 5B).

Interestingly, even though it has been reported that the repetitive stimulation of Tregs can lead to the loss of FOXP3 expression, our expanded cells acquired a stable phenotype of Treg cells, probably due to the inclusion of a resting period of 3 days involving the interruption of the continuous signal through the TCR [50], or by prolonged treatment with RAPA that involves the inhibition of both mTOR1 and mTOR2 pathways, favoring the maintenance of FOXP3 expression [51].

### Example 5

### Evaluation of Allospecific Induced Regulatory T Cell stability in the Presence of Pro-Inflammatory Cytokines

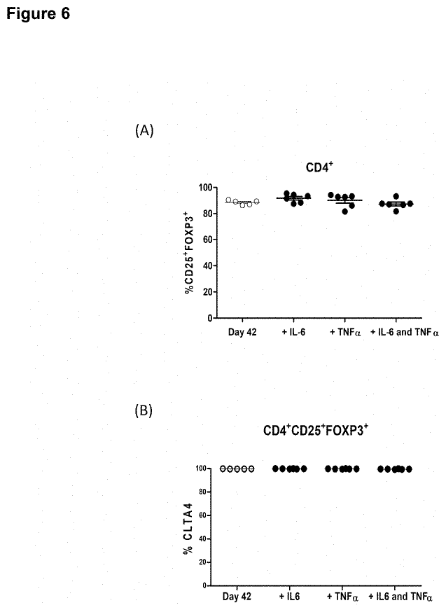

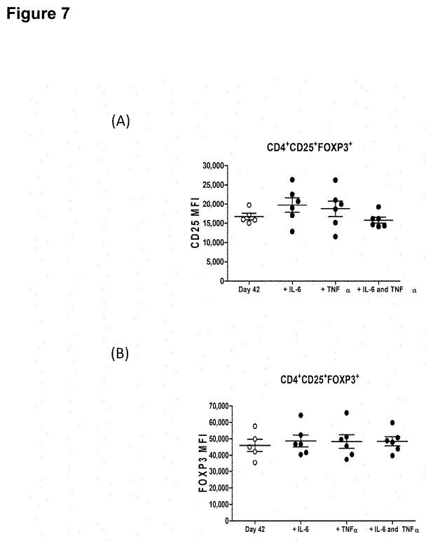

To evaluate the stability of allospecific iTregs assays, on day 28 of expansion, iTregs were stimulated for two additional rounds of stimulation/resting cycles in the presence or absence of 10 ng/mL of IL-6 or TNF-α, using the same stimuli (anti-CD3/anti-CD28 beads IL-2, TGF-β and RAPA) indicated in example 4. No differences were observed in the percentage of CD25+ FOXP3+ cells (FIG. 6A) or the levels of CTLA-4+ (FIG. 6B), CD25 (FIG. 7A) and FOXP3 expression (FIG. 7B), which indicates that the cells obtained with the method of the present invention are resistant to the pro-inflammatory effects of IL-6 and TNF-α.

It has been reported, using an experimental autoimmune encephalomyelitis model, that iTregs are sensitive to the effect of TNF-α, inducing AKT activation and reducing the phosphorylation of TGFβ1-induced SMAD3 and therefore a lower binding of phosphorylated SMAD3 to the promoter region of the FOXP3 gene [52]. In this context, the inhibition of the PI3K/AKT/mTOR pathway by RAPA could contribute to the stable phenotype observed in our expanded iTregs. Finally, it has been reported that the treatment of iTregs with IL-6 does not affect neither the expression of FOXP3 nor its suppressive activity in vitro compared to thymic Tregs, This was explained by the low expression of IL-6 receptor in iTregs, which is downregulated by both IL-2 and TGF-β1, suggesting that iTregs might be more stable in an inflammatory environment [26].

### Example 6

### Expanded Allospecific iTregs Suppression Assay

Ten days before the suppression assay, DCs (the donor was the source of the DCs used in the allogeneic co-culture of example 1) were derived from CD14+ monocytes following the protocol mentioned in example 1. On the day of the suppression assay, DCs were washed, irradiated and resuspended in OpTmizer™ CTS™ T-Cell Expansion culture medium (Gibco). Next, CD3+ T cells (the donor was the source of allospecific iTregs) were separated using MACS columns, which were subsequently labeled with CFSE and co-cultured for 4 days with the CTV-labeled iTregs, in the presence of dendritic cells from donor 2.

CTV labelling of iTregs allowed to discriminate this population from proliferating CD3+ T cells (which lose CFSE) at the time of data analysis. CD3+ T cells and dendritic cells were in ratios of 4 to 1. The ratios of iTregs cells versus CD3+ T cells used in the co-cultures were 1:2, 1:8 and 1:32. At the end of the culture, the cells were stained with anti-CD4 and anti-CD8 antibodies, and acquired on the Attune® NxT flow cytometer (Life Technologies). The percentage of allo-specific proliferation of CD4+ and CD8+ T cells (responder T cells) in the presence and absence of iTregs was determined by dilution of the CFSE marker. The percentage of suppression was calculated using the following formula: [(Proliferation of Tresp without Tregs−Proliferation of Tresp with Tregs)/Proliferation of Tresp without Tregs]×100. As negative controls of the assay, T cells that did not proliferate in the co-cultures (CD4+ CFSE− cells) and Mo-DCs from another individual (3rd+iTregs alo) were included.

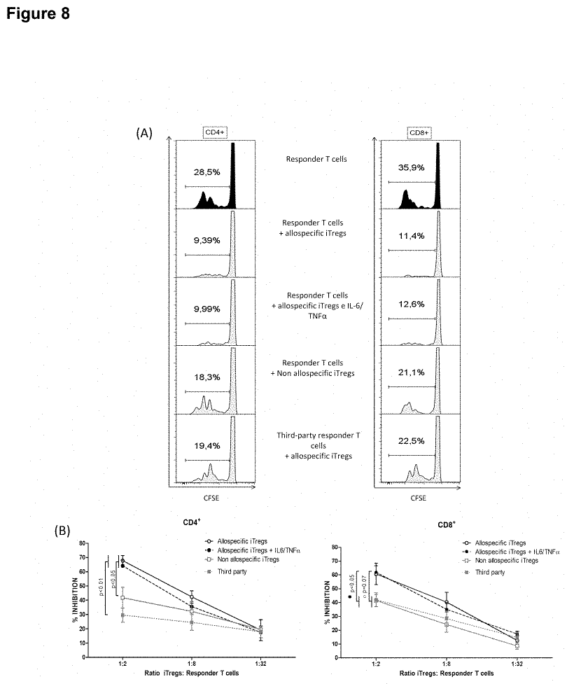

CD3+ T cells were able to proliferate in the presence of allogeneic Mo-DCs (only responder T cells). The expanded allospecific iTregs (iTregs allo) suppressed the proliferation of CD3+ alloreactive T cells only when they were stimulated with the DCs from their respective donors, but did not suppress alloreactive T cells generated with DCs from a different individual (third party) (FIG. 8A, histograms), indicating that the suppression is antigen-specific. These results indicate that, most likely, responses to other antigens, such as bacterial or viral, would not be affected by the iTregs. Furthermore, the addition of pro-inflammatory cytokines (IL-6 and TNF-α) in the cultures did not alter the suppressive capacity of allospecific iTregs (FIG. 8B). This suggests that once the iTregs are infused, they would maintain their function under conditions of inflammation, for example as a consequence of transplantation or after infection during the post-transplant period, supporting their potential use as adoptive therapy.

### Example 7

### Cytokine Production Assay

The levels of cytokines IL-2, IL-10 and IFN-γ were measured in the supernatants from the cultures of the suppression assays by flow cytometry using the immunoassay kit LEGENDplex (Biolegend), according to the manufacturer's guidelines.

Briefly, the supernatants were incubated with a panel of capture beads, then mixed with biotinylated detection antibodies and subsequently with streptavidin-phycoerythrin (SA-PE), emitting fluorescent signals with intensities in proportion to the concentration of cytokine present in the supernatant, which were quantified using the Attune® NxT flow cytometer (Thermo Fisher Inc). The concentration of the cytokines in the supernatants was determined using a standard curve generated in the same assay. The experiments were carried out in quadruplicate and repeated twice. The following conditions were considered: 1) only responder T cells activated for 5 days with anti-CD3/CD28 beads and 2) co-culture of responder T cells activated with the beads and in the presence of autologous Tregs in a 2:1 ratio.

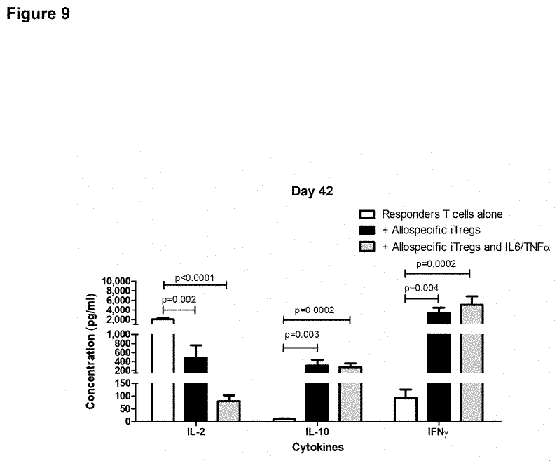

It has been reported that the suppression exerted by Tregs can affect different responses including cell proliferation, effector function, and differentiation from conventional cells to effector cells, as well as the amplitude of their effector function. On the other hand, the suppression of proliferation may involve direct contact between the Treg and the effector cell or the antigen presenting cells, through co-inhibitory receptors such as CTLA-4 and PD-1, or affect the consumption of IL-2 by the responder cell [53]. The iTregs expanded for 6 weeks obtained in the present invention showed a high expression of CTLA-4 (FIG. 6B), which is important for Treg to decrease the stimulatory capacity of DCs after their interaction with CD80/CD86 molecules expressed on DCs [54], as well as to restrain the activation of “naive” T cells by competing with CD28 for its binding to CD80/CD86. Furthermore, in the supernatants of the suppression assays, a reduction of IL-2 was observed, which is indicative of the mechanism of metabolic disruption exerted by the Tregs, inhibiting the response of conventional T cells [55]. Finally, an increase in the production of IL-10 and IFN-γ was detected; the first is considered an immunosuppressive cytokine that acts by regulating the function of APCs and inhibiting the proliferation of T cells [53] (FIG. 9). Although IFN-γ is considered a pro-inflammatory cytokine, evidence indicates its immunoregulatory role. It has been proposed that IFNγ could influence Treg function via the induction of chemokine receptors such as CXCR3, which would promote their effective migration to the target organ or by inducing FOXP3 expression in “naive” T cells, probably via STAT1 [56].

### Example 8

### Evaluation of Induced Allospecific Regulatory T Cell Stability

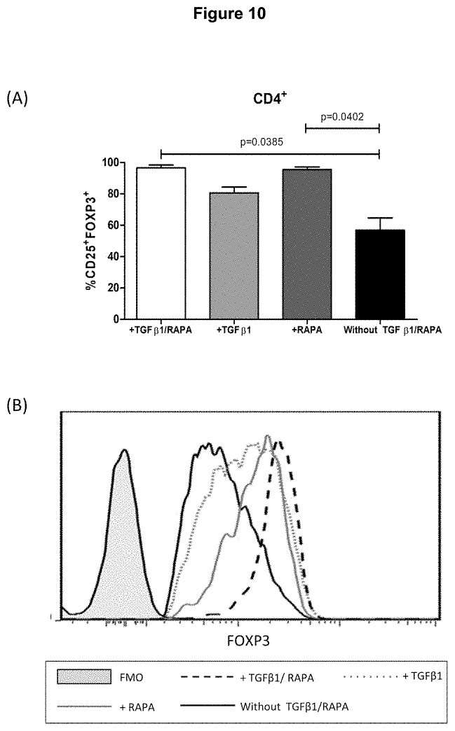

In the sixth week of expansion, the allospecific iTregs were cultured for an additional week in the presence or absence of TGF-β1 and/or RAPA. The concentration of the other stimuli (anti-CD3/CD28 beads and IL-2) and the expansion/resting scheme were the same as those described in example 4. According to the results, the maintenance of the CD25 and FOXP3 phenotype (FIG. 10A) and FOXP3 levels (FIG. 10B) of the expanded iTregs require the presence of RAPA, which indicates that this agent should be considered in a possible clinical use of the iTregs.

### REFERENCES

1. Game, D. S. and R. I. Lechler, Pathways of allorecognition: implications for transplantation tolerance. Transpl Immunol, 2002. 10(2-3): p. 101-8.
2. Lechler, R. I., et al., Organ transplantation—how much of the promise has been realized? Nat Med, 2005. 11(6): p. 605-13.
3. Lechler, R. I., O. A. Garden, and L. A. Turka, The complementary roles of deletion and regulation in transplantation tolerance. Nat Rev Immunol, 2003. 3(2): p. 147-58.
4. Golshayan, D. and M. Pascual, Tolerance-Inducing Immunosuppressive Strategies in Clinical Transplantation. Drugs, 2008.
5. Ferrer, I. R., et al., Induction of transplantation tolerance through regulatory cells: from mice to men. Immunol Rev, 2014. 258(1): p. 102-16.
6. Sakaguchi, S., et al., Regulatory T cells and immune tolerance. Cell, 2008. 133(5): p. 775-87.
7. Brunkow, M. E., et al., Disruption of a new forkhead-winged-helix protein, scurfin, results in the fatal lymphoproliferative disorder of the scurfy mouse. nature genetics, 2001.
8. Bennett, C. L., et al., The immune dysregulation, polyendocrinopathy, enteropathy, X-linked syndrome (IPEX) is caused by mutations of FOXP3. Nat Genet, 2001. 27(1): p. 20-1.
9. Curotto de Lafaille, M. A. and J. J. Lafaille, Natural and adaptive foxp3+ regulatory T cells: more of the same or a division of labor? Immunity, 2009. 30(5): p. 626-35.
10. Miyao, T., et al., Plasticity of Foxp3(+) T cells reflects promiscuous Foxp3 expression in conventional T cells but not reprogramming of regulatory T cells. Immunity, 2012. 36(2): p. 262-75.
11. Polansky, J. K., et al., DNA methylation controls Foxp3 gene expression. Eur J Immunol, 2008. 38.
12. Hoffmann, P., et al., Only the CD45RA+ subpopulation of CD4+CD25high T cells gives rise to homogeneous regulatory T-cell lines upon in vitro expansion. Blood, 2006. 108(13): p. 4260-7.
13. Mandapathil, M., et al., Isolation of functional human regulatory T cells (Treg) from the peripheral blood based on the CD39 expression. J Immunol Methods, 2009. 346(1-2): p. 55-63.
14. Wang, R., et al., Expression of GARP selectively identifies activated human FOXP3+ regulatory T cells. Proc Natl Acad Sci USA, 2009. 106(32): p. 13439-44.
15. Liu, W., et al., CD127 expression inversely correlates with FoxP3 and suppressive function of human CD4+ T reg cells. J Exp Med, 2006. 203(7): p. 1701-11.
16. Kleinewietfeld, M., et al., CD49d provides access to “untouched” human Foxp3+ Treg free of contaminating effector cells. Blood, 2009. 113(4): p. 827-36.
17. Salgado, F. J., et al., CD26: a negative selection marker for human Treg cells. Cytometry A, 2012. 81(10): p. 843-55.
18. Hoffmann, P., et al., Loss of FOXP3 expression in natural human CD4+CD25+ regulatory T cells upon repetitive in vitro stimulation. Eur J Immunol, 2009. 39(4): p. 1088-97.
19. Tang, Q. and F. Vincenti, Transplant trials with Tregs: perils and promises. J Clin Invest, 2017. 127(7): p. 2505-2512.
20. Dons, E. M., et al., Induced regulatory T cells: mechanisms of conversion and suppressive potential. Hum Immunol, 2012. 73(4): p. 328-34.
21. Josefowicz, S. Z., et al., Extrathymically generated regulatory T cells control mucosal TH2 inflammation. Nature, 2012. 482(7385): p. 395-9.
22. Samstein, R. M., et al., Extrathymic generation of regulatory T cells in placental mammals mitigates maternal-fetal conflict. Cell, 2012. 150(1): p. 29-38.
23. Haribhai, D., et al., A requisite role for induced regulatory T cells in tolerance based on expanding antigen receptor diversity. Immunity, 2011. 35(1): p. 109-22.
24. Haribhai, D., et al., A central role for induced regulatory T cells in tolerance induction in experimental colitis. J Immunol, 2009. 182(6): p. 3461-8.
25. Maganto-Garcia, E., et al., Foxp3+-inducible regulatory T cells suppress endothelial activation and leukocyte recruitment. J Immunol, 2011. 187(7): p. 3521-9.
26. Zheng, S. G., J. Wang, and D. A. Horwitz, Cutting edge: Foxp3+CD4+CD25+ regulatory T cells induced by IL-2 and TGF-beta are resistant to Th17 conversion by IL-6. J Immunol, 2008. 180(11): p. 7112-6.
27. Huter, E. N., et al., Cutting edge: antigen-specific TGF beta-induced regulatory T cells suppress Th17-mediated autoimmune disease. J Immunol, 2008. 181(12): p. 8209-13.
28. Kong, N., et al., Antigen-specific transforming growth factor β-induced Treg cells, but not natural Treg cells, ameliorate autoimmune arthritis in mice by shifting the Th17/Treg cell balance from Th17 predominance to Treg cell predominance. Arthritis Rheum, 2012. 64(8): p. 2548-58.
29. Inomata, T., et al., Impaired Function of Peripherally Induced Regulatory T Cells in Hosts at High Risk of Graft Rejection. Sci Rep, 2016. 6: p. 39924.
30. Yue, X., et al., Control of Foxp3 stability through modulation of TET activity. J Exp Med, 2016. 213(3): p. 377-97.
31. Wang, J., T. W. J. Huizinga, and R. E. M. Toes, De novo generation and enhanced suppression of human CD4+CD25+ regulatory T cells by retinoic acid. J Immunol, 2009. 183(6): p. 4119-26.
32. Hippen, K., et al., Generation and large-scale expansion of human inducible regulatory T cells that suppress graft-versus-host disease. Am J Transplant, 2011. 11(6): p. 1148-57.
33. Hsu, P., et al., IL-10 Potentiates Differentiation of Human Induced Regulatory T Cells via STAT3 and Foxo1. J Immunol, 2015. 195(8): p. 3665-74.
34. Schmidt, A., et al., Comparative Analysis of Protocols to Induce Human CD4+Foxp3+ Regulatory T Cells by Combinations of IL-2, TGF-beta, Retinoic Acid, Rapamycin and Butyrate. PLoS One, 2016. 11(2).
35. Candia, E., et al., Single and combined effect of retinoic acid and rapamycin modulate the generation, activity and homing potential of induced human regulatory T cells. 2017. 12(7): p. e0182009.
36. Tu, W., et al., Efficient generation of human alloantigen-specific CD4+ regulatory T cells from naive precursors by CD40-activated B cells. Blood, 2008. 112(6): p. 2554-62.
37. Fujii, S., et al., The linkage of innate to adaptive immunity via maturing dendritic cells in vivo requires CD40 ligation in addition to antigen presentation and CD80/86 costimulation. J Exp Med, 2004. 199(12): p. 1607-18.
38. Jonuleit, H., et al., Induction of interleukin 10-producing, nonproliferating CD4(+) T cells with regulatory properties by repetitive stimulation with allogeneic immature human dendritic cells. J Exp Med, 2000. 192(9): p. 1213-22.
39. Litjens, N. H., et al., Allogeneic Mature Human Dendritic Cells Generate Superior Alloreactive Regulatory T Cells in the Presence of IL-15. J Immunol, 2015. 194(11): p. 5282-93.
40. Cools, N., et al., Immunosuppression induced by immature dendritic cells is mediated by TGF-beta/IL-10 double-positive CD4+ regulatory T cells. J Cell Mol Med, 2008. 12(2): p. 690-700.
41. Banerjee, D. K., et al., Expansion of FOXP3(high) regulatory T cells by human dendritic cells (DCs) in vitro and after injection of cytokine-matured DCs in myeloma patients. Blood, 2006. 108(8): p. 2655-61.
42. Zheng, J., et al., CD40-activated B cells are more potent than immature dendritic cells to induce and expand CD4(+) regulatory T cells. Cell Mol Immunol, 2010. 7(1): p. 44-50.
43. Yao, Z., et al., Nonredundant roles for Stat5a/b in directly regulating Foxp3. Blood, 2007. 109(10): p. 4368-75.
44. Xiao, S., et al., Retinoic acid increases Foxp3+ regulatory T cells and inhibits development of Th17 cells by enhancing TGF-beta-driven Smad3 signaling and inhibiting IL-6 and IL-23 receptor expression. J Immunol, 2008. 181(4): p. 2277-84.
45. Hill, J. A., et al., Retinoic acid enhances Foxp3 induction indirectly by relieving inhibition from CD4+CD44hi Cells. Immunity, 2008. 29(5): p. 758-70.
46. Putnam, A. L., et al., Clinical grade manufacturing of human alloantigen-reactive regulatory T cells for use in transplantation. Am J Transplant, 2013. 13(11): p. 3010-20.
47. Veerapathran, A., et al., Ex vivo expansion of human Tregs specific for alloantigens presented directly or indirectly. Blood, 2011. 118(20): p. 5671-80.
48. Camperio, C., et al., Forkhead transcription factor FOXP3 upregulates CD25 expression through cooperation with RelA/NF-kappaB. PLoS One, 2012. 7(10): p. e48303.
49. Tang, Q. and K. Lee, Regulatory T-cell therapy for transplantation: how many cells do we need? Curr Opin Organ Transplant, 2012. 17(4): p. 349-54.
50. Sauer, S., et al., T cell receptor signaling controls Foxp3 expression via PI3K, Akt, and mTOR. Proc Natl Acad Sci USA, 2008. 105(22): p. 7797-802.
51. Delgoffe, G. M., et al., The kinase mTOR regulates the differentiation of helper T cells through the selective activation of signaling by mTORC1 and mTORC2. Nat Immunol, 2011. 12(4): p. 295-303.
52. Zhang, Q., et al., TNF-alpha impairs differentiation and function of TGF-beta-induced Treg cells in autoimmune diseases through Akt and Smad3 signaling pathway. J Mol Cell Biol, 2013. 5(2): p. 85-98.
53. Schmidt, A., N. Oberle, and P. Krammer, Molecular Mechanisms of Treg-Mediated T Cell Suppression. Frontiers in Immunology, 2012. 3(51).
54. Wing, K., et al., CTLA-4 control over Foxp3+ regulatory T cell function. Science, 2008. 322(5899): p. 271-5.
55. Pandiyan, P., et al., CD4+CD25+Foxp3+ regulatory T cells induce cytokine deprivation-mediated apoptosis of effector CD4+ T cells. Nat Immunol, 2007. 8(12): p. 1353-62.
56. Nishibori, T., et al., Impaired development of CD4+ CD25+ regulatory T cells in the absence of STAT1: increased susceptibility to autoimmune disease. J Exp Med, 2004. 199(1): p. 25-34.

1. A subpopulation of induced regulatory human T cells comprising a phenotype CD25⁺ECTLA-4⁺FOXP3⁺, allospecific and stable in the presence of proinflammatory cytokines.

2. The subpopulation of induced regulatory according with claim 1, wherein the proinflammatory cytokines are TNF-α and IL-6.

3. The subpopulation of induced regulatory human T cells according to claim 1, wherein the allospecificity is evaluated by the suppression of proliferation and cytokine production of donor CD3⁺ T cells.

4. A method for in vitro generation and expansion of regulatory T cells according to claim 1, comprising, at the 6th week of expansion, reaching an expansion of 23×10⁴ times the initial number, wherein 90% of cells are CD25⁺ECTLA-4⁺FOXP3⁺, and giving rise to 4,600 million allospecific iTregs from 20,000 naïve T cells.

5. The method for in vitro generation and expansion of regulatory T cells according to claim 4, further comprising:
a. generating allospecific Tregs from co-cultures between “naïve” T cells from an individual (donor 1) and Immature Dendritic Cells derived from monocytes (Mo-DCs) from another individual (donor 2);
b. isolating iTregs obtained in the previous step; and
c. polyclonally expanding the isolated iTregs obtained from the previous step.

6. The method according with claim 5, wherein in step a), Mo-DCs are derived from peripheral blood monocytes after culture for 9 to 10 days in the presence of 50 ng/mL GM-CSF and 50 ng/mL IL-4, and were identified by low levels of surface MHC Class II and expression of costimulatory molecules.

7. The method according with claim 5, wherein in step a), the co-culture is performed using a 10:1 ratio (Naive:immature Dendritic Cells)

8. The method according with claim 5, wherein the co-culture of step a) is performed for a period of 8 to 10 days in the presence of 5 to 10 ng/ml TGF-β1, 10 nM ATRA and 50 to 100 U/ml IL-2.

9. The method according with claim 5, wherein in the isolation of step b) proliferating allospecific iTregs are sorted on the base of CD25^{hi}.

10. The method according with claim 5, wherein expansion in step c) is performed for 6 weeks, with anti-CD3/CD28 beads, at a ratio of from 1:1 to 1:2 beads per cell, in the presence of 5 to 10 ng/mL TGF-β1, of 50 to 100 U/mL IL-2 and 100 ng/mL RAPA for 4 days.

11. The method according with claim 10, wherein at day 4 of expansion, beads are removed from culture and cells are left alone in culture media containing 50 U/mL of IL-2 for 3 days.

12. The method according with claim 11, further comprising expansion/resting cycles repeated for 6 weeks.

13. A method for reducing the use of immunosuppressors in a patient in need thereof comprising using the subpopulation of induced regulatory human T cells according to claim 1 for induction of transplantation tolerance in the patient in need thereof and as an alternative or complementary therapy to the use of immunosuppressors.
